(19) United States
(12) Patent Application Publication
(10) Pub. No.: US 2002/0001800 A1
(43) Pub. Date: Jan. 3, 2002

(54) DIAGNOSTIC METHODS USING SERIAL TESTING OF POLYMORPHIC LOCI

(76) Inventor: STANLEY N. LAPIDUS, BEDFORD, NH (US)

Correspondence Address: TESTA, HURWITZ & THIBEAULT, LLP, HIGH STREET TOWER, 125 HIGH STREET, BOSTON, MA, 02110, US

(21) Appl. No.: 09/333,206

(22) Filed: Jun. 15, 1999

Related U.S. Application Data
(60) Provisional application No. 60/096,588, filed on Aug. 14, 1998.

(51) Int. Cl. C12Q 1/68; C12P 19/34

(52) U.S. Cl. 435/6; 435/91.1

(57) ABSTRACT

Methods are provided for assaying the heterozygosity status of an individual member of a population. Methods of the invention are useful for detecting loss of heterozygosity in a nucleic acid sample. Methods of the invention are particularly useful for identifying individuals with mutations indicative of cancer.

### FIELD OF THE INVENTION

This invention relates to methods for analyzing polymorphic loci in cellular samples. Methods of the invention are useful in disease diagnosis. Methods of the invention are especially useful in minimizing the number of steps involved in a diagnostic assay.

### BACKGROUND OF THE INVENTION

Many polymorphic genetic loci exist. A genetic locus is polymorphic when individuals in a population possess a plurality of genotypes at the locus. Many polymorphic loci differ in only a single nucleotide. Other polymorphic loci contain larger genotypic changes such as inversions, translocations, insertions, or deletions, including differences in the number of minisatellite or microsatellite tandem repeats. An individual member of a population is homozygous at a given polymorphic locus when both alleles at that locus are identical. Conversely, an individual is heterozygous at a given genetic locus when the two alleles at that locus are different. Typically, an individual member of a population is homozygous at a subset of the polymorphic loci, and heterozygous at the remaining polymorphic loci. The heterozygosity status of an individual can be a useful indicator of disease.

The presence of heterozygosity in a biological sample can be used as a general indicator of genomic integrity. For example, loss of heterozygosity indicates that a first allele is underepresented relative to a second allele, typically due to deletion of the first allele. Loss of heterozygosity at a genetic locus is often indicative of disease. In particular, loss of heterozygosity is often associated with cancer. The genomic instability that is characteristic of cancer is thought to arise from a coincident disruption of genomic integrity and a loss of cell cycle control mechanisms. Generally, a disruption of genomic integrity is thought merely to increase the probability that a cell will engage in the multistep pathway leading to cancer. However, coupled with a loss of cell cycle control mechanisms, a disruption in genomic integrity may be sufficient to generate a population of genomically unstable neoplastic cells. Loss of heterozygosity is a common genetic change characteristic of the early stages of such transformation. Loss of heterozygosity at a number of tumor suppressor genes has been implicated in tumorigenesis. For example, loss of heterozygosity at the P53 tumor suppressor locus has been correlated with various types of cancer. Ridanpaa, et al., Path. Res. Pract, 191: 399-402 (1995). The loss of the apc and dcc tumor suppressor genes has also been associated with tumor development. Blum, Europ. J. Cancer, 31A: 1369-372 (1995).

Loss of heterozygosity in an individual is therefore a potentially useful indicator of disease, and is especially useful for detecting the early stages of diseases such as cancer. However, different individuals in a population are heterozygous at different loci. There is therefore a need in the art for efficient and inexpensive methods to identify a heterozygous locus in an individual member of a population, and to assay the heterozygous locus for loss of heterozygosity.

### SUMMARY OF THE INVENTION

The invention provides methods for a highly-sensitive diagnostic assay involving the interrogation of only a small number of genetic loci. According to the invention, a minimal number of genetic loci are examined in a patient sample in order to identify a locus that is useful for further diagnostic analysis. In one embodiment of the invention, a heterozygous locus is identified, and subsequently interrogated for any indication of loss of heterozygosity.

In one embodiment, the present invention provides methods for detecting indicia of disease in a biological sample by serially analyzing different genetic loci. In a preferred embodiment, methods of the invention are useful for identifying a heterozygous locus, and determining whether loss of heterozygosity has occurred at that locus. Accordingly, preferred methods of the invention comprise sequentially analyzing a plurality of genetic loci that are known or suspected to be polymorphic in a population. A first locus is analyzed in a patient sample to determine if it is heterozygous or homozygous. If the first locus is homozygous, a second locus is analyzed to determine its zygosity status. This process is repeated until a heterozygous locus is identified in the sample. Preferably, once a heterozygous locus is identified it is used for subsequent analysis to detect a mutation, for example, a loss of heterozygosity at the locus.

Methods of the invention significantly reduce the labor involved in the detection of mutation (e.g., a deletion (including a loss of heterogygosity), addition, substitution, rearrangement, or other nucleic acid change).

In a preferred embodiment of the invention, an assay is performed to detect a genomic disruption using the first of a series of polymorphic loci that is determined to be heterozygous. Thus, it is not necessary to conduct the assay on every polymorphic locus known or suspected to be associated with a disease or with a genetic abnormality. In a more preferred embodiment, a plurality of single base polymorphic loci are analyzed serially in a biological sample until one such locus is found to be heterozygous. A number of a first allele and a number of a second allele are then determined for the heterozygous locus. The two numbers are compared. A statistically significant difference between the numbers is indicative of a mutation in at least some of the cells in the sample. Such a mutation is indicative of a disruption in genomic stability that may be associated with disease, especially cancer. According to methods of the invention, patients who are diagnosed as having a mutation at a heterozygous locus may be screened using other, more invasive techniques.

Accordingly, in a preferred embodiment, methods of the invention comprise selecting a plurality of polymorphic loci in a genetic region that is known to be associated with a disease (e.g. several polymorphisms within the p53 region). Members of this predetermined plurality of polymorphisms are tested sequentially in a patient sample, as described above, until a heterozygous locus is identified.

In further embodiment, a predetermined plurality of polymorphic loci may be selected for each of several different genetic regions (e.g. polymorphisms in the p53, dcc, and acc regions). In a first step, a first polymorphic locus from each plurality is tested to determine whether it is heterozygous. Subsequent polymorphic loci from each set are tested until

In an alternative embodiment, a predetermined plurality of polymorphic loci contains one or more polymorphic loci from each of several different genetic regions. According to methods of the invention, the polymorphic loci are tested sequentially in a patient sample until a heterozygous locus is identified. According to this embodiment, the heterozygous locus may be in any one of the several genetic regions.

A preferred polymorphic locus is a locus that is heterozygous in a high percentage of the population, preferably in over 10% of the population, more preferably in about 50% of the population. According to methods of the invention, a heterozygous locus will generally be identified in fewer steps by analyzing a series of polymorphic loci that are heterozygous in a high percentage of the population as opposed to a series of loci that are heterozygous in only a small subset of the population.

In a preferred embodiment, a predetermined set or plurality of polymorphic loci contains a number of loci sufficient to ensure (with at least 50%, preferably 90%, and most preferably 99% certainty) that a heterozygous locus will be identified in a patient sample according to methods of the invention. In a most preferred embodiment, a plurality of polymorphic loci comprises seven polymorphic loci.

Methods of the invention are useful for detecting a mutation, such as loss of heterozygosity, that is indicative of a disease such as cancer. Methods of the invention are especially useful for detecting mutations in a subpopulation of cells in a heterogeneous biological sample. In a preferred embodiment, methods of the invention are used to detect mutations in nucleic acids in blood, biopsy tissue, sputum, pus, semen, saliva, lymph, cerebrospinal fluid, urine, or stool, most preferably a cross-section or circumferential-section of stool. Methods of the invention a particularly useful for detecting early signs of colorectal cancer in a small subpopulation of cells in a patient's stool sample.

In a preferred embodiment, methods of enumerating alleles comprise enumerating a single nucleotide corresponding to a first allele at a heterozygous polymorphic locus; and enumerating a single nucleotide corresponding to a second allele at the locus. Enumeration is preferably carried out by using radiolabeled allele-specific probes. In a preferred embodiment, a radiolabeled allele-specific probe specifically hybridizes to a region containing an allele of the heterozygous polymorphic locus. In a more preferred embodiment, enumeration is accomplished using single base extension of an oligonucleotide probe. Single base extension is accomplished by hybridizing an oligonucleotide probe upstream of the single base polymorphic nucleotide to be detected, and extending the probe (via polymerase) using radiolabeled nucleotides, preferably chain-terminating nucleotides, such as dideoxynucleotides, that are complementary to the nucleotide to be detected. Other detection moieties, such as molecular weight labels, impedance tags, florescent tags, and the like can be used.

Preferred radioisotopes include ³⁵S, ³²P, ³H, ¹²⁵I, and ¹⁴C. If two different radiolabels are used, the first and second labels (corresponding to first and second alleles) are distinguished by their different characteristic emission spectra. The number of radioactive decay events is measured for each oligonucleotide without separating the two oligonucleotide from each other. In alternative embodiments, allele specific probes are separated from each other prior to enumeration.

In a further embodiment, the invention also comprises identifying one or more heterozygous loci that can be used in a series of diagnostic assays for an individual. For example, the same heterozygous locus or loci can be used in yearly assays for loss of heterozygosity.

In a preferred embodiment of the invention, a heterozygous locus is identified for a patient, and the locus is then used in a series of assays for loss of heterozygosity. For example, samples from different tissues may be interrogated for loss of heterozygosity using the same heterozygous locus. Alternatively, the same heterozygous locus may be interrogated on a regular basis (e.g. a yearly basis) in order to detect a deletion which may be indicative of a disease such as cancer. The invention therefore provides methods for identifying patient specific diagnostic markers.

In an alternative embodiment, sequential or serial analysis methods of the invention are also useful to detect, in an individual, the presence of a mutation associated with a disease. For example, a disease may be known to be associated with any one of a plurality of mutations. According to methods of the invention, an individual suspected of having the disease is tested serially for the presence of each member of the plurality of mutations, until the presence of one of the mutations is detected. Upon detection of one of the plurality of mutations, the individual is diagnosed as having the disease. Upon such a diagnosis, information about the presence of any of the remaining mutations is redundant. Therefore, once one of the mutations has been detected, the individual does not need to be tested for the presence of any additional mutations.

### DETAILED DESCRIPTION OF THE INVENTION

In general, the invention provides methods for identifying a heterozygous genetic locus that are analyzed to detect a mutation in one of the two alleles at the locus. A mutation in one of the alleles is identified by detecting fewer numbers of one allele relative to the other allele in a biological sample. Methods of the invention are particularly useful to detect loss of heterozygosity in a biological sample.

The invention provides methods for optimizing or minimizing the number of steps involved in identifying a diagnostically useful heterozygous locus in an individual member of a population. Methods of the invention involve a serial or sequential analysis of potentially heterozygous loci in an individual until a locus that is heterozygous in that individual is identified. Accordingly, once a heterozygous locus is identified, no additional genetic loci need be analyzed. Therefore, serial analysis according to the invention minimizes the total number of genetic loci that need to be interrogated. Methods of the invention generally involve the analysis of only a subset of the loci that would otherwise have to be analyzed.

Methods of the invention therefore minimize the amount of material (oligonucleotides, gels, radioisotopes) required to identify a heterozygous locus. In a preferred embodiment, a serial detection method is automated to repeat the step of determining heterozygosity at a series of genetic loci. According to this method, genetic loci belonging to a predetermined group of potentially heterozygous loci are analyzed until a heterozygous locus is identified. In a more preferred embodiment, the process is automated to perform a serial analysis on multiple samples, each sample obtained from a different individual.

A heterozygous locus is particularly useful for disease diagnosis if a deletion of one of the two alleles is correlated with disease. For example, a polymorphism in a tumor suppressor gene is useful to detect a mutation in the tumor suppressor which may be associated with cancer. Deletions, and particularly deletions characteristic of loss of heterozygosity, typically involve several hundreds to several thousands of base pairs (and up to several million base pairs). Any one of the heterozygous genetic loci within the deleted genetic region can be used to detect the deletion. Therefore, in a preferred embodiment of the invention, sequential analysis is performed on a series of polymorphic loci belonging to a genetic region that is suspected of being deleted in a diseased individual. Preferred genetic regions include tumor suppressor genes such as p53, dcc, and acc.

In one embodiment of the invention, once a heterozygous genetic locus has been identified by serial analysis of a patient sample, an assay is performed to determine whether there is a deletion or other mutation in one of the alleles at the locus. In a preferred embodiment, a number of a first allele is counted and compared to a number of a second allele. A statistically significant difference between the numbers of the first and second alleles is indicative of a deletion of one of the alleles. Methods of the invention are useful to detect a deletion in a subpopulation of cells (or cellular debris) in a heterogeneous biological sample including both wild-type cells and deletion-containing cells (or debris therefrom). Methods of the invention are particularly useful to detect loss of heterozygosity in a subpopulation of cells.

Methods of the invention are also useful for RNA analysis. Methods of the invention can be used to identify a heterozygous locus in an expressed region of the genome. Subsequent enumerative analysis compares the expression level of a first allele relative to a second allele at the heterozygous locus. Accordingly, methods of the invention are useful to detect increased expression of an allele associated with disease. For example, methods of the invention may be used to detect increased expression of an oncogene allele (e.g. ras, fos, jun, myc, myb, or other oncogenes), which is indicative of cancer. Alternatively, methods of the invention are useful to detect decreased expression of an allele associated with disease (e.g. decreased expression of a tumor suppressor allele). As discussed above, methods of the invention can detect changes in allele expression in a subpopulation of cells in a heterogeneous biological sample.

1. Detecting a Heterozygous Locus Using Serial Analysis of Single Nucleotide Polymorphic Loci

The following analysis exemplifies methods of the invention using single nucleotide polymorphisms that are 50% heterozygous. A similar analysis may be applied to other types of polymorphic loci that are present at different frequencies in the population. In the following example of serial analysis, heterozygous loci are identified in most patient samples by examining two to four loci, and often by examining only one locus. This is in contrast to a standard assay which examines many loci in a single step. In the following example, at least seven loci need to be analyzed simultaneously in a standard assay to be 99% certain that a heterozygous locus will be identified.

In preferred methods for detecting loss of heterozygosity (LOH), a single nucleotide polymorphism (SNP), for which an individual is heterozygous, is used to distinguish the two alleles (the maternal and paternal alleles) at a genetic locus. Useful SNPs are preferably about 50% heterozygous. That is, at a particular SNP locus, an individual has a 50% chance of being heterozygous and a 50% chance of being homozygous.

SNPs are spaced roughly every 1,000 to 10,000 base pairs in the human genome. Deletions which are characteristic of loss of heterozygosity are much larger than this spacing; typically such deletions are at least one megabase and up to tens of megabases in length. Accordingly, there are many candidate SNPs in each region of deletion characteristic of LOH.

SNPs that are spaced sufficiently far apart sort independently. That is, zygosity status for a particular SNP is not influenced by the zygosity status of adjacent SNPs. SNPs for which a given patient is heterozygous are said to be “informative” for that patient, and loss of heterozygosity can be determined at such SNP loci. According to methods of the invention, a single heterozygous SNP is sufficient to assay for loss of heterozygosity.

Assuming that SNPs are 50% heterozygous, and sort independently, the probability that all “X” SNPs at a given locus are homozygous is represented by the equation:

=½^{x} (I)

For a confidence level greater that 99% that at least one tested SNP is heterozygous, at least seven SNPs are needed, as shown by the calculation: ½⁷=0.0078125, which is less than 1% (½⁶=1.56%).

Accordingly, for a patient who has never been screened before it must be determined which of the seven possible loci to probe. Conventional methods dictate two distinct approaches:

In one procedure, LOH tests are run on all seven loci in parallel. For example, in a situation in which there are 100 patients, 700 LOH tests would need to be run in order to ensure, with 99% confidence, that at lest one heterozygous SNP will be identified per patient.

In an alternative method, a gel or blot is run and probed for all seven markers prior to running the LOH assay. The results of the gel guide the selection of which particular SNP will be analyzed. Although more than one SNP can be heterozygous, it is only necessary to examine one. This procedure is advantageous over the first approach (above) in that it is simpler to run a single gel or blot for all seven possible heterozygous SNPs than to run the LOH test seven times. For 100 patients, 100 gels or blots and 100 LOH tests would be run. Using known sample preparation, running such a gel or blot would require seven capture probes and seven PCRs.

However, the present invention simplifies the task of determining LOH even further. The methods of the present invention embody a testing strategy that is aided by the fact that extremely rapid test procedures are generally not required and timeliness of intervention is less critical. Most patients (roughly 99% or more in a regularly screened population) are negative and follow-up treatment is reserved for only the patients who test positive.

In the present invention, a biological sample is tested for the first of seven predetermined SNPs. If the results of this analysis indicate that the patient is heterozygous, the testing stops, and the degree of LOH is determined. If the patient is homozygous at that locus, the next SNP is tested, and so on until either a heterozygous site is identified or until all seven SNPs have been tested.

While it is true that for some patients, it may be necessary to test five, six or even seven polymorphisms, for half of the patients it will be sufficient to stop after testing the first SNP. For 75% of the patients, it will be sufficient to stop after analyzing the second polymorphism. On average, it will only be necessary to test two SNPs per patient. That is, for 100 patients, only about 200 SNPs will need to be interrogated. Thus, the present invention provides the surprising advantage that serial testing of polymorphic loci provides a tremendous reduction in the overall testing volume (i.e., the number of hybrid captures and the number of PCRs).

A further unexpected result of the present invention is that the average number of two SNPs per patient is constant whether it is seven loci or seven hundred loci that need to be investigated. The spreadsheet provided in Table 1 illustrates this point. At each round, 50% of the number of assays are heterozygous (and no further analysis needs to be done), and 50% of the patients need at least one more round of testing. Table 1 shows that, for 1,000 patients, this process asymptotes to twice the number of samples, no matter how many polymorphisms there are to be tested.

TABLE 1
Assuming 1000 patients:

| | |
|---|---|
| Number of patients which undergo first round: | 1,000.000 |
| Number of patients which undergo second round: | 500.000 |
| Number of patients which undergo third round: | 250.000 |
| Number of patients which undergo fourth round: | 125.000 |
| etc. | 62.500 |
| | 31.250 |
| | 15.625 |
| | 7.813 |
| | 3.906 |
| | 1.953 |
| | 0.977 |
| | 0.488 |
| | 0.244 |
| | 0.122 |
| | 0.061 |
| | 0.031 |
| | 0.015 |
| | 0.008 |
| total number assays performed | 1,999.992 |

In alternative embodiments of the invention, other polymorphic loci (e.g. deletions, insertions, variations in mini- or micro-satellite repeat numbers) are used in addition to, or instead of, single nucleotide polymorphisms. A polymorphism that is less than 50% heterozygous is also useful for methods of the invention. In a preferred embodiment, a polymorphism is at least 10% heterozygous. If a predetermined set of polymorphisms contains polymorphisms having different heterozygosity frequencies in the population, the higher frequency polymorphic loci are preferably tested before the lower frequency polymorphic loci. For most patient samples, one of the higher frequency polymorphic loci will be heterozygous, and the lower frequency polymorphic loci will not need to be examined.

2. Determination of Heterozygosity

The heterozygosity of a given genetic locus may be determined using methods known in the art. In a preferred embodiment, genomic nucleic acid is prepared from a patient sample, for example from a blood sample. An amount of genomic nucleic acid is digested with a restriction enzyme, electrophoresed on an agarose gel, and transferred to a membrane by Southern blotting. Alternatively, an amount of genomic nucleic acid is dot-blotted onto a membrane. Membrane bound genomic nucleic acid is exposed to detectably-labeled allele-specific hybridization probes. In one embodiment, different allele-specific probes are labeled with differentially detectable labels (e.g. different fluorescent tags or different radio-isotopes). In an alternative embodiment, different allele-specific probes, labeled with the same detectable label, are hybridized to genomic DNA in separate reactions. Hybridization conditions are chosen to prevent non-specific hybridization. Hybridization is quantified for each allele-specific probe. If only one probe hybridizes to the genomic DNA, the patient is homozygous at that locus. If about the same level of hybridization is observed for two allele-specific probes, the patient is heterozygous at that locus. Other methods for detecting heterozygosity (including RFLP and mini- or micro-satellite analysis) are known in the art.

In one embodiment of the invention, genomic nucleic acid encompassing a polymorphic locus is amplified prior to further analysis. In another embodiment of the invention, nucleic acids are sheared or cut into small fragments by, for example, by restriction digestion. Single-stranded nucleic acid fragments may be prepared using well-known methods. See, e.g., Sambrook, et al., Molecular Cloning, A Laboratory Manual (1989) incorporated by reference herein.

3. Allele Detection Using Single Base Extension

A preferred method of testing for the presence of a single-nucleotide variant, or for quantifying single-nucleotide variants, is to conduct a single base extension assay. Such an assay is performed by annealing an oligonucleotide primer to a complementary nucleic acid, and extending the 3′ end of the annealed primer with a chain terminating nucleotide that is added in a template directed reaction catalyzed by, for example, a DNA polymerase. The selectivity and sensitivity of a single base primer extension reaction are affected by the length of the oligonucleotide primer and the reaction conditions (e.g. annealing temperature, salt concentration). Alternatively, gaps between the 3′ end of the primer and the single base to be detected may be filled in by primer extension using unlabelled nucleotides. This works best if the single base(s) to be detected is (are) unique within the extended primer sequence.

The selectivity of a primer extension reaction reflects the amount of exact complementary hybridization between an oligonucleotide primer and a nucleic acid in a sample. A highly-selective reaction promotes primer hybridization only to nucleic acids with an exact complementary sequence (i.e. there are no base mismatches between the hybridized primer and nucleic acid). In contrast, in a non-selective reaction, the primer also hybridizes to nucleic acids with a partial complementary sequence (i.e. there are base mismatches between the hybridized primer and nucleic acid). In general, parameters which favor selective primer hybridization (for example shorter primers and higher annealing temperatures) result in a lower level of hybridized primer. Therefore, parameters which favor a selective single-base primer extension assay result in decreased sensitivity of the assay.

In a preferred method of the invention at least two cycles of a single-base extension reaction are conducted. By repeating the single-base extension reaction, the signal of a single-base primer extension assay is increased without reducing the selectivity of the assay. Cycling increases the signal, and the extension reaction can therefore be performed under highly selective conditions (for example, the primer is annealed at about or above its Tm).

In a preferred embodiment, detection methods are performed by annealing an excess of primer under conditions which favor exact hybridization, extending the hybridized primer, denaturing the extended primer, and repeating the annealing and extension reactions at least once. In a most preferred embodiment, the reaction cycle comprises a step of heat denaturation, and the polymerase is temperature stable (for example, Taq polymerase or Vent polymerase).

Preferred primer lengths are between 10 and 100 nucleotides, more preferably between 10 and 50 nucleotides, and most preferably about 30 nucleotides. Useful primers are those that hybridize adjacent a suspected mutation site, such that a single base extension at the 3′ end of the primer incorporates a nucleotide complementary to the allele-specific nucleotide if it is present on the template.

Preferred hybridization conditions comprise annealing temperatures about or above the Tm of the oligonucleotide primer in the reaction. The Tm of an oligonucleotide primer is determined by its length and GC content, and is calculated using one of a number of formulas known in the art. Under standard annealing conditions, a preferred formula for a primer approximately 25 nucleotides long, is Tm (° C.)=4×(Number of Gs+Number of Cs)+2×(Number of As+Number of Ts).

In a preferred reaction, the annealing and denaturation steps are performed by changing the reaction temperature. In one embodiment of the invention, the primer is annealed at about the Tm for the primer, the temperature is raised to the optimal temperature for extension, the temperature is then raised to a denaturing temperature. In a more preferred embodiment of the invention, the reaction is cycled between the annealing temperature and the denaturing temperature, and the single base extension occurs during transition from annealing to denaturing conditions.

In a preferred detection means, two or more cycles of extension are performed. In a more preferred means, between 5 and 100 cycles are performed. In a further embodiment, between 10 and 50 cycles, and most preferably about 30 cycles are performed.

In a preferred embodiment of the invention, the nucleotide added to the 3′ end of the primer in a template dependent reaction is a chain terminating nucleotide, for example a dideoxynucleotide. In a more preferred embodiment, the nucleotide is detectably labeled.

Detection methods of the invention may comprise conducting at least two cycles of single-base extension with a segmented primer. In a preferred embodiment, the segmented primer comprises a short first probe and a longer second probe capable of hybridizing to substantially contiguous portions of the target nucleic acid. The two probes are exposed to a sample under conditions that do not favor the hybridization of short first probe in the absence of longer second probe. Factors affecting hybridization are well known in the art and include temperature, ion concentration, pH, probe length, and probe GC content. A first probe, because of its small size, hybridizes numerous places in an average genome. For example, any given 8-mer occurs about 65,000 times in the human genome. However, an 8-mer has a low melting temperature (Tₘ) and a single base mismatch greatly exaggerates this instability. A second probe, on the other hand, is larger than the first probe and will have a higher Tₘ. A 20-mer second probe, for example, typically hybridizes with more stability than an 8-mer. However, because of the small thermodynamic differences in hybrid stability generated by single nucleotide changes, a longer probe will form a stable hybrid but will have a lower selectivity because it will tolerate nucleotide mismatches. Accordingly, under unfavorable hybridization conditions for the first probe (e.g., 10-40▪C above first probe Tₘ), the first probe hybridizes with high selectivity (i.e., hybridizes poorly to sequence with even a single mismatch), but forms unstable hybrids when it hybridizes alone (i.e., not in the presence of a second probe). The second probe will form a stable hybrid but will have a lower selectivity because of its tolerance of mismatches.

The extension reaction will not occur absent contiguous hybridization of the first and second probes. A first (proximal) probe alone is not a primer for template-based nucleic acid extension because it will not form a stable hybrid under the reaction conditions used in the assay. Preferably, the first probe comprises between about 5 and about 10 nucleotides. The first probe hybridizes adjacent to a nucleic acid suspected to be mutated. A second (distal) probe in mutation identification methods of the invention hybridizes upstream of the first probe and to a substantially contiguous region of the target (template). The second probe alone is not a primer of template-based nucleic acid extension because it comprises a 3′ non-extendible nucleotide. The second probe is larger than the first probe, and is preferably between about 15 and about 100 nucleotides in length.

Template-dependent extension takes place only when a first probe hybridizes next to a second probe. When this happens, the short first probe hybridizes immediately adjacent to the site of the suspected single base mutation. The second probe hybridizes in close proximity to the 5′ end of the first probe. The presence of the two probes together increases stability due to cooperative binding effects. Together, the two probes are recognized by polymerase as a primer. This system takes advantage of the high selectivity of a short probe and the hybridization stability imparted by a longer probe in order to generate a primer that hybridizes with the selectivity of a short probe and the stability of a long probe. Accordingly, there is essentially no false priming with segmented primers. Since the tolerance of mismatches by the longer second probe will not generate false signals, several segmented primers can be assayed in the same reaction, as long as the hybridization conditions do not permit the extension of short first probes in the absence of the corresponding longer second probes. Moreover, due to their increased selectivity for target, methods of the invention may be used to detect and identify a target nucleic acid that is available in small proportion in a sample and that would normally have to be amplified by, for example, PCR in order to be detected.

By requiring hybridization of the two probes, false positive signals are reduced or eliminated. As such, the use of segmented oligonucleotides eliminates the need for careful optimization of hybridization conditions for individual probes, as presently required in the art, and permits extensive multiplexing. Several segmented oligonucleotides can be used to probe several target sequences assayed in the same reaction, as long as the hybridization conditions do not permit stable hybridization of short first probes in the absence of the corresponding longer second probes.

The first and second probes hybridize to substantially contiguous portions of the target. For purposes of the present invention, substantially contiguous portions are those that are close enough together to allow hybridized first and second probes to function as a single probe (e.g., as a primer of nucleic acid extension). Substantially contiguous portions are preferably between zero (i.e., exactly contiguous so there is no space between the portions) nucleotides and about one nucleotide apart. A linker is preferably used where the first and second probes are separated by two or more nucleotides, provided the linker does not interfere with the assay (e.g., nucleic acid extension reaction). Such linkers are known in the art and include, for example, peptide nucleic acids, DNA binding proteins, and ligation. It has now been realized that the adjacent probes bind cooperatively so that the longer, second probe imparts stability on the shorter, first probe. However, the stability imparted by the second probe does not overcome the selectivity (i.e., intolerance of mismatches) of the first probe. Therefore, methods of the invention take advantage of the high selectivity of the short first probe and the hybridization stability imparted by the longer second probe.

Thus, first and second probes preferably are hybridized to substantially contiguous regions of target, wherein the first probe is immediately adjacent and upstream of a polymorphic site, for example, a single nucleotide polymorphism. The sample is then exposed to dideoxy nucleic acids that are complements of possible allele nucleotides. Deoxynucleotides may alternatively be used if the reaction is stopped after the addition of a single nucleotide. Polymerase, either endogenously or exogenously supplied, catalyzes incorporation of a dideoxy base on the first probe.

Alternatively, a segmented oligonucleotide comprises a series of first probes, wherein sufficient stability is only obtained when all members of the segmented oligonucleotide simultaneously hybridize to substantially contiguous portions of a nucleic acid. Although short probes exhibit transient, unstable hybridization, adjacent short probes bind cooperatively and with greater stability than each individual probe. Together, a series of adjacently-hybridized first probes will have greater stability than individual probes or a subset of probes in the series. For example, in an extension reaction with a segmented primer comprising a series of three first probes (i.e., three short probes with no terminal nucleotide capable of hybridizing to a substantially contiguous portion of a nucleic acid upstream of the target nucleic acid), the concurrent hybridization of the three probes will generate sufficient cooperative stability for the three probes to prime nucleic acid extension and the short probe immediately adjacent to a polymorphic site will be extended. Thus, segmented probes comprising a series of short first probes offer the high selectivity (i.e., intolerance of mismatches) of short probes and the stability of longer probes.

Several cycles of extension reactions preferably are conducted in order to amplify the assay signal. Extension reactions are conducted in the presence of an excess of first and second probes, labeled dNTPs or ddNTPs, and heat-stable polymerase. Once an extension reaction is completed, the first and second probes bound to target nucleic acids are dissociated by heating the reaction mixture above the melting temperature of the hybrids. The reaction mixture is then cooled below the melting temperature of the hybrids and first and second probes permitted to associate with target nucleic acids for another extension reaction. In a preferred embodiment, 10 to 50 cycles of extension reactions are conducted. In a most preferred embodiment, 30 cycles of extension reactions are conducted.

Labeled ddNTPs or dNTPs preferably comprise a “detection moiety” which facilitates detection of the extended primers, or extended short first probes in a segmented primer reaction. Detection moieties are selected from the group consisting of fluorescent, luminescent or radioactive labels, enzymes, haptens, molecular weight markers, impedance markers, and other chemical tags such as biotin which allow for easy detection of labeled extension products. Fluorescent labels such as the dansyl group, fluorescein and substituted fluorescein derivatives, acridine derivatives, coumarin derivatives, pthalocyanines, tetramethylrhodamine, Texas Red®, 9-(carboxyethyl)-3-hydroxy-6-oxo-6H-xanthenes, DABCYL® and BODI PY® (Molecular Probes, Eugene, Oreg.), for example, are particularly advantageous for the methods described herein. Such labels are routinely used with automated instrumentation for simultaneous high throughput analysis of multiple samples.

In a preferred embodiment, primers or first probes comprise a “separation moiety.” Such separation moiety is, for example, hapten, biotin, or digoxigenin. These primers or first probes, comprising a separation moiety, are isolated from the reaction mixture by immobilization on a solid-phase matrix having affinity for the separation moiety (e.g., coated with anti-hapten, avidin, streptavidin, or anti-digoxigenin). Non-limiting examples of matrices suitable for use in the present invention include nitrocellulose or nylon filters, glass beads, magnetic beads coated with agents for affinity capture, treated or untreated microtiter plates, and the like.

In a preferred embodiment, the separation moiety is incorporated in the labeled ddNTPs or dNTPs. By denaturing hybridized primers or probes, and immobilizing primers or first probes extended with a labeled ddNTP or dNTP to a solid matrix, labeled primers or labeled first probes are isolated from unextended primers or unextended first probes and second probes, and primers or first probes extended with an unlabeled ddNTPs by one or more washing steps.

In an alternative preferred embodiment, the separation moiety is incorporated in the primers or first probes, provided the separation moiety does not interfere with the first primer's or probe's ability to hybridize with template and be extended. Eluted primers or first probes are immobilized to a solid support and can be isolated from eluted second probes by one or more washing steps.

Alternatively, the presence of primers or first probes that have been extended with a labeled terminal nucleotide may be determined without eluting hybridized primers or probes. The methods for detection will depend upon the label or tag incorporated into the primers or first probes. For example, radioactively labeled or chemiluminescent first probes that have bound to the target nucleic acid can be detected by exposure of the filter to X-ray film. Alternatively, primers or first probes containing a fluorescent label can be detected by excitation with a laser or lamp-based system at the specific absorption wavelength of the fluorescent reporter.

In an alternative embodiment, the bound primers or first and second probes are eluted from a matrix-bound target nucleic acid (see below). Elution may be accomplished by any means known in the art that destabilizes nucleic acid hybrids (i.e., lowering salt, raising temperature, exposure to formamide, alkali, etc.). In a preferred embodiment, the bound oligonucleotide probes are eluted by incubating the target nucleic acid-segmented primer complexes in water, and heating the reaction above the melting temperature of the hybrids.

Deoxynucleotides may be used as the detectable single extended base in any of the reactions described above that require single base extension. However, in such methods, the extension reaction must be stopped after addition of the single deoxynucleotide. Moreover, the extension reaction need not be terminated after the addition of only one deoxynucleotide if only one labeled species of deoxynucleotide is made available in the sample for detection of the single base polymorphism. This method may actually enhance signal if there is a nucleotide repeat including the interrogated single base position.

In a preferred embodiment, target nucleic acids are immobilized to a solid support prior to exposing the target nucleic acids to primers or segmented primers and conducting an extension reaction. Once the nucleic acid samples are immobilized, the samples are washed to remove non-immobilized materials. The nucleic acid samples are then exposed to one or more set of primers or segmented primers according to the invention. Once the single-base extension reaction is completed, the primers or first probes extended with a labeled ddNTP or dNTP are preferably isolated from unextended probes and probes extended with an unlabeled ddNTPs or dNTP. Bound primers or first and second probes are eluted from the support-bound target nucleic acid. Elution may be accomplished by any means known in the art that destabilizes nucleic acid hybrids (i.e., lowering salt, raising temperature, exposure to formamide, alkali, etc.). In a preferred embodiment, the first and second probes bound to target nucleic acids are dissociated by incubating the target nucleic acid-segmented primer complexes in water, and heating the reaction above the melting temperature of the hybrids and the extended first probes are isolated. In an alternative preferred embodiment, the extension reaction is conducted in an aqueous solution. Once the single-base extension reaction is completed, the oligonucleotide probes are dissociated from target nucleic acids and the extended first probes are isolated. In an alternative embodiment, the nucleic acids remain in aqueous phase.

In a preferred embodiment, the separation moiety is incorporated in the labeled ddNTPs or dNTPs. By immobilizing eluted primers or first probes extended with a labeled ddNTP or dNTP to a solid support, labeled primers or first probes are isolated from unextended first probes and second probes, and primers or first probes extended with an unlabeled ddNTPs by one or more washing steps.

In an alternative preferred embodiment, the separation moiety is incorporated in the primers or first probes, provided the separation moiety does not interfere with the first primer's or probe's ability to hybridize with template and to be extended. Eluted primers or first probes are immobilized to a solid support and can be isolated from eluted second probes by one or more washing steps.

Finally, methods of the invention comprise isolating and sequencing the extended first probes. A “separation moiety” such as, for example, hapten, biotin, or digoxigenin is used for the isolation of extended first probes. In a preferred embodiment, first probes comprising a separation moiety are immobilized to a solid support having affinity for the separation moiety (e.g., coated with anti-hapten, avidin, streptavidin, or anti-digoxigenin). Non-limiting examples of supports suitable for use in the present invention include nitrocellulose or nylon filters, glass beads, magnetic beads coated with agents for affinity capture, treated or untreated microtiter plates, and the like.

According to methods of the invention, the amount of each allele at a heterozygous locus in a patient sample is quantified. In a preferred embodiment, the alleles are quantified by enumeration. A number of the first allele and a number of the second allele are counted. The numbers are counted as described in U.S. Pat. No. 5,670,325 or in U.S. Ser. No. 08/876,857, the disclosures of which are incorporated herein by reference. Briefly, the number of detectable moieties that are incorporated in the base extension reactions are counted. If the detection moieties are impedance balls, they are counted using an impedance counter such as a Coulter counter. If the detection moieties are radioisotopes, they are counted by converting the number of radioactive decay events (measured using a scintillation counter for example) into a number of molecules using a known number of decay events per molecule.

Either portions of a coding strand or its complement may be detected in methods according to the invention. In a preferred embodiment, both first and second strands of an allele are present in a sample during hybridization to an oligonucleotide probe. The sample is exposed to an excess of probe that is complementary to a portion of the first strand, under conditions to promote specific hybridization of the probe to the portion of the first strand. In a most preferred embodiment, the probe is in sufficient excess to bind all the portion of the first strand, and to prevent reannealing of the first strand to the second strand of the allele. Also in a preferred embodiment, the second strand of an allele is removed from a sample prior to hybridization to an oligonucleotide probe that is complementary to a portion of the first strand of the allele.

4. Enumerative Analysis

In one embodiment of the invention, the numbers of molecules of each allele of a heterozygous locus in a biological sample are compared using a statistical analysis. In a preferred embodiment, methods of the invention involve a comparison of the number of molecules of two nucleic acids that are expected to be present in the sample in equal numbers in normal (non-mutated) cells. In a preferred embodiment, the comparison is between (1) an amount of a first allele at a heterozygous locus and (2) an amount of a second allele at the heterozygous locus A statistically-significant difference between the amounts of the two genomic polynucleotide segments indicates that a mutation, for example loss of heterozygosity, has occurred in at least a subpopulation of the alleles in the sample. Loss of heterozygosity can result in loss of either allele, the important information is the presence or absence of a statistically significant difference between the number of molecules of each allele in the sample. If an allele sequence is amplified, as in the case of certain oncogene mutations, the detected amount of the amplified allele is greater than the detected amount of wild-type by a statistically-significant margin.

Statistically-significant difference between numbers of first and second alleles at a heterozygous locus obtained from a biological sample may be determined by any appropriate method. See, e.g., Steel, et al., Principles and Procedures of Statistics, A Biometrical Approach (McGraw-Hill, 1980), the disclosure of which is incorporated by reference herein. An exemplary method is to determine, based upon a desired level of specificity (tolerance of false positives) and sensitivity (tolerance of false negatives) and within a selected level of confidence, the difference between numbers of first and second alleles that must be obtained in order to reach a chosen level of statistical significance.

What is claimed is:

1. A method for detecting indicia of disease in a biological sample, the method comprising the steps of:
(a) serially analyzing members of a plurality of polymorphic loci until a member of said plurality is determined to be a heterozygous locus;
(b) determining a first number of a first allele of said heterozygous locus;
(c) determining a second number of a second allele of said heterozygous locus; and
(d) determining whether a statistically-significant difference exists between said first and second numbers, the presence of said statistically-significant difference being indicative of the presence of a disease.

2. The method of claim 1, wherein said biological sample is a stool sample.

3. The method of claim 2, wherein said stool sample comprises a cross-section of stool.

4. The method of claim 1, wherein said biological sample is selected from the group consisting of blood, biopsy tissue, sputum, pus, semen, saliva, lymph, cerebrospinal fluid, and urine.

5. The method of claim 1, wherein said predetermined plurality of polymorphic loci is selected from the group consisting of polymorphic loci in the p53, dcc, and acc genes.

6. The method of claim 1, wherein said polymorphic loci are 50% heterozygous in a population from which the biological sample was obtained.

7. The method of claim 1, wherein said predetermined plurality of polymorphic loci comprises seven polymorphic loci.

8. A method for detecting a deletion in a biological sample, the method comprising the steps of:
(a) serially analyzing members of a predetermined plurality of polymorphic loci until a member of said plurality is determined to be a heterozygous locus in said biological sample;
(b) determining a first number of a first allele of said heterozygous locus;
(c) determining a second number of a second allele of said heterozygous locus; and
(d) determining whether a statistically-significant difference exists between said first and second numbers, the presence of said statistically-significant difference being indicative of the presence of a deletion.

9. The method of claim 1, wherein said determining steps comprise exposing said biological sample to at least one allele-specific oligonucleotide probe.

10. The method of claim 9, wherein said probe is detectably labeled.

11. The method of claim 10, wherein said label is a radioisotope.

12. The method of claim 9, wherein said sample is exposed to two different allele-specific probes, each having a different detectable label.

13. The method of claim 1, wherein said disease is cancer.

14. The method of claim 13, wherein said cancer is colorectal cancer.

15. A method for detecting an informative genetic locus in a biological sample, the method comprising serially analyzing individual members of a predetermined plurality of genetic loci until a member of said plurality that is heterozygous is identified.
